(19) United States
(12) Patent Application Publication
(10) Pub. No.: US 2007/0106290 A1
(43) Pub. Date: May 10, 2007

(54) Conformable electrode catheter and method of use

(76) Inventors: Thomas A. Turano, Arlington, MA (US); Omar Amirana, Boston, MA (US)

Correspondence Address: Kirkpatrick & Lockhart Preston Gates Ellis LLP, STATE STREET FINANCIAL CENTER, One Lincoln Street, BOSTON, MA, 02111-2950, US

(21) Appl. No.: 11/516,379

(22) Filed: Sep. 6, 2006

Related U.S. Application Data
(60) Provisional application No. 60/734,559, filed on Nov. 8, 2005.

(51) Int. Cl. A61B 18/14

(52) U.S. Cl. 606/41; 606/49

(57) ABSTRACT

An apparatus and method for closing the tunnel of a patent foramen ovale (PFO) including the steps of advancing a device, including an energy delivery element, in the lumen of the tunnel of the PFO, energizing the energy delivery element, and withdrawing the energized energy delivery element from the second end of the lumen of the tunnel toward the first end of the lumen of the PFO tunnel, thereby substantially sealing the tissues in the tunnel of the PFO.

### RELATED APPLICATIONS

This application claims priority to and benefit of U.S. provisional application 60/734,559 filed on Nov. 8, 2005, the entire content of which is incorporated by reference herein.

### FIELD OF THE INVENTION

The invention relates to the field of radio-frequency (RF) medical devices in general and more specifically to the field of treating intracardiac defects with an energy source.

### BACKGROUND OF THE INVENTION

The human heart is divided into four compartments or chambers. The left and right atria are located in the upper portion of the heart and the left and right ventricles are located in the lower portion of the heart. The left and right atria are separated from each other by a muscular wall, the interatrial septum, and the ventricles are separated by the interventricular septum.

Either congenitally or by acquisition, abnormal openings (holes or shunts) can occur between the chambers of the heart or between the great vessels, causing inappropriate blood flow. Such deformities are usually congenital and originate during fetal life when the heart forms from a folded tube into a four chambered, two-unit, i.e., atrial and ventricular, system. The septal deformities result from the incomplete formation of the septum, or muscular wall, between the left and right chambers of the heart and can cause significant problems.

One such septal deformity or defect, a patent foramen ovale (PFO), is a persistent tunnel with a flap-like opening in the wall between the right atrium and the left atrium of the heart. Since left atrial pressure is normally higher than right atrial pressure, the flap typically stays closed. Under certain conditions, however, right atrial pressure exceeds left atrial pressure, creating the possibility for right to left shunting of venous blood that can allow blood clots and other toxins to enter the systemic circulation. This is particularly problematic for patients who have deep vein thrombosis or clotting abnormalities.

Referring to FIG. 1A, a unipolar RF medical device such as an RF scalpel known to the prior art includes radiofrequency (RF) generator 2 having a first electrode 4 connected to the medical device 6 such as a scalpel. A ground plate 10 placed on the patient is also connected to RF generator 2. The RF generator 2 also includes an earth ground 8. When an RF voltage is applied to the device 6, current is induced to flow (arrow I) between the device 6 and the ground plate 10. The point of contact of the medical device 6 produces a high RF energy concentration with a correspondingly high density current. The high RF energy concentration generates heat in the immediate tissue causing an alteration in the tissue.

Referring to FIG. 1B, in another embodiment known to the prior art, the return electrode 8′ of the RF generator 2 is not connected to earth ground but instead is placed in close proximity to the first electrode 4 of the medical device 6. Current flow is again induced between the first 4 and return 8′ RF electrodes and again the high RF energy concentration near the tip of the medical device 6, causes tissue heating and alteration.

Such prior art devices can be used to close the PFO in the heart. The problem arises that the topology of tissues in the heart varies from person to person. Thus, for an electrode with a small contact area, only “spot welds” could be achieved. These “spot welds” do not provide extended closure of the entire surface area of the PFO. For an electrode with a larger contact area, a good electrode-tissue contact is difficult to achieve, which could hinder complete closure of the PFO. The present invention provides a solution to these problems.

### SUMMARY OF THE INVENTION

The invention in one aspect relates to an apparatus for closing the tunnel of a PFO. In one embodiment, the apparatus includes a catheter having a proximal end and a distal end and a pod disposed at the distal end of the catheter. The pod includes a conformable conductive tissue contacting surface. The conformable conductive tissue contacting surface of the pod substantially uniformly contacts the surface of the cardiac tissues adjacent to the entrance of the tunnel to deliver energy to substantially close the PFO.

Another aspect the invention relates to a method for closing the tunnel of a PFO. In one embodiment, the method includes the steps of advancing a device, including an energy delivery element, in the lumen of the tunnel of the PFO from a first end of the lumen of the tunnel toward a second end of the lumen of the tunnel. Next, the method includes the step of energizing the energy delivery element and withdrawing the energized energy delivery element while the energy delivery element is continuously or intermittently energized from the second end of the lumen of the tunnel toward the first end of the lumen of the PFO tunnel, thereby substantially sealing the tissues in the tunnel of the PFO from the second end of the tunnel to the first end of the tunnel.

In yet another aspect, the invention relates to a method for closing the tunnel of a PFO using an apparatus including a catheter having a proximal end and a distal end and a pod disposed at the distal end of the catheter. The pod includes a conformable tissue contacting surface. The pod is placed such that the conformable tissue contacting surface of the pod substantially uniformly contacts the surface of the cardiac tissues adjacent to the entrance of the tunnel and RF energy is delivered to the PFO to substantially close the PFO.

As used throughout, to “substantially seal” or “substantially close” the PFO it is meant that a stable tissue bridge will be formed across the PFO, which will withstand physiological pressures. A substantially closed or sealed PFO, however, may still have one or more small gaps or openings which will in at least some cases close over time via the healing process.

### BRIEF DESCRIPTION OF THE DRAWINGS

In the drawings like reference characters generally refer to the same parts throughout the different views. Also, the drawings are not necessarily to scale, emphasis instead generally being placed upon illustrating the principles of the invention.

These and further aspects of the invention can be better understood with reference to the attached specification and drawings in which:

FIG. 1A is a block diagram of a unipolar RF medical device as known to the prior art;

FIG. 1B is a block diagram of a bipolar RF medical device as known to the prior art;

FIG. 2 is a perspective cutaway view of a heart illustrating a PFO.

FIG. 3 is a highly schematic cross-sectional diagram of a unipolar embodiment of the apparatus of the invention;

FIG. 4A illustrates a portion of the flexible member including an inflatable RF pod in a collapsed position according to an illustrative embodiment of the invention.

FIG. 4B illustrates a portion of the flexible member illustrated in FIG. 4A including an inflatable RF pod in an expanded position according to an illustrative embodiment of the invention.

FIGS. 5A-5C depict the embodiment of the invention of FIG. 3, being positioned and deployed against a surface in the heart;

FIG. 6 is a highly schematic cross-sectional diagram of a bi-polar embodiment of the invention; and

FIGS. 7A-7E depict the embodiment of the invention of FIG. 6 being used to close a PFO.

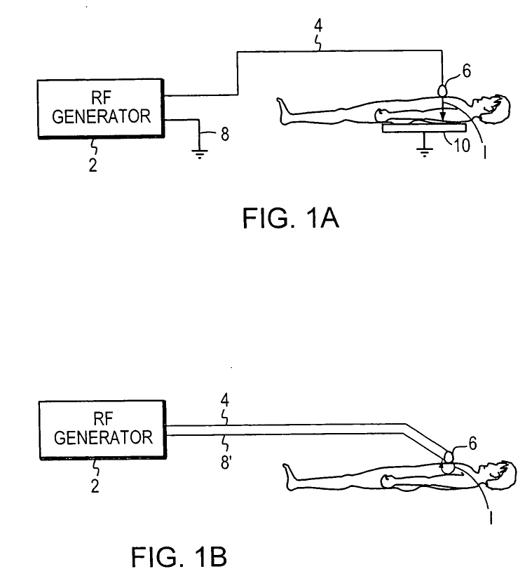

### DESCRIPTION OF A PREFERRED EMBODIMENT

The present invention features systems and related methods for closing cardiac openings, such as, for example, the PFO, described below. Throughout the description, the terms proximal and distal refer to the position of elements relative to the operator of the exemplary medical device. Proximal is that portion of the medical device closer to the operator and distal is that portion of the medical device further away from the operator.

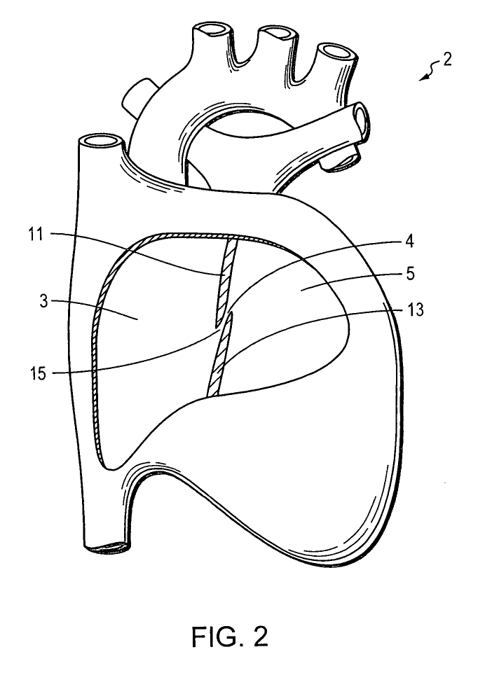

FIG. 2 depicts a cutaway view of a heart 2. The heart 2 includes a septum 4 that divides a right atrium 3 from a left atrium 5. The septum 4 includes a septum secundum 11 and a septum primum 13. An exemplary cardiac opening, a patent foramen ovale 15, that is to be corrected by the system and related method of the present invention is located between the septum secundum 11 and the septum primum 13. The PFO 15 provides an undesirable fluid communication between the right atrium 3 and the left atrium 5 and, under certain conditions, allows for the shunting of blood and toxins carried by the blood between the right atrium 3 and the left atrium 5. If the PFO 15 is not closed or obstructed in some manner, a patient is placed at higher risk for an embolic stroke, in addition to other circulatory abnormalities.

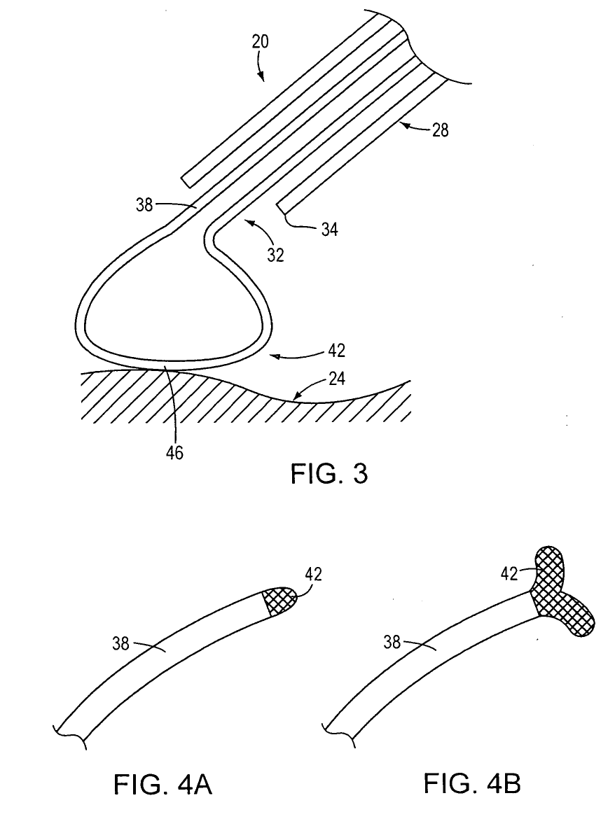

In a brief overview, and referring to FIG. 3, a generalized unipolar embodiment of the apparatus 20 of the invention is depicted. This embodiment includes a delivery catheter portion 28 and an RF electrode portion 32. The RF electrode portion includes a flexible member 38 and an RF or electrode pod 42 positioned at the distal end of the flexible member 38. In one embodiment, the flexible member 38 is conductive. The RF pod 42 has a flexible, generally bulbous shape with a conformable surface 46. In one embodiment of the invention, the conformable surface 46 is conductive. In one embodiment, the RF pod 42 is made from conductive materials or a conformable form material embedded with conductive materials. For example, the RF pod 42 may be made from a hydrogel blended with conductive materials, or a non-woven fabric such as cotton embedded with conductive materials, or a metallic material with a flexible chain-link design that enables the electrode to conform to the anatomical topography structure of the right atrium.

In another embodiment, the RF pod 42 may be made from plastic, thermoplastic elastomer, or other elastomeric material with metallic filing or a metallic coating on its outer surface. For example, the RF pod 42 may be made of gold-filled silicone, or metal-coated polyethylene.

Referring now to FIGS. 4A-4B, alternatively, the RF pod 42 may be made from conductive expandable material on the outside surface and saline or gel enclosed within the pod 42. Saline or a gel can be used as the conductor to deliver RF energy to the conductive expandable outer surface. Alternatively, saline or gel may be injected into the conductive expandable RF pod 42 after the pod has been positioned at the cardiac site for treatment. In a particular embodiment, the RF pod 42 is a conductive sponge, for example, carbon filled silicone.

Referring back to FIG. 3, in one embodiment according to the invention, the conformable surface 46 is conductive while the RF electrode portion 32, including the flexible member 38, is not conductive. As the RF pod 42 is inflated by an inflation medium such as, for example, saline or a gel, RF energy is applied and current flows through the conductive inflation medium to the conductive conformable surface 46, through the cardiac surface 24 to the ground (not shown).

The RF pod 42 transitions reversibly between a collapsed position illustrated, for example, in FIG. 4A and an expanded position illustrated, for example, in FIG. 4B. In its collapsed configuration illustrated in FIG. 4A, the circumference of the RF pod 42 is substantially similar to the outer circumference of the flexible member 38. In its expanded position, the RF pod 42 expands to an expanded configuration, e.g., a substantially bulbous configuration illustrated, for example, in FIG. 4B. In this substantially bulbous configuration, the RF pod 42 is conformable when applied to the surface contour of the treatment site in the right atrium or within the tunnel of the PFO. Through its conductive surface 46, the RF pod 42 delivers RF energy to the cardiac tissues and to the tissues within the tunnel of the PFO.

Referring back to FIG. 3, in yet another embodiment, the RF pod 42 may include a plurality of pores (not shown) on its conformable surface 46. Saline or other conductive media is used to inflate the RF pod 42. As the RF pod 42 is inflated, the conductive media weeps from through the pores of the conformable surface 46 of the RF pod 42 thereby creating a conductive media interface between the conformable surface 46 and the cardiac tissues 24. In this embodiment, the conductive media serves as the conductor of RF energy to the cardiac tissues.

According to the embodiments of the invention described herein, the expandable RF pod 42 has the advantage of avoiding the formation of coagulum or blood clots at effective yet moderate levels of RF energy. In addition, the expandable RF pod 42 is soft and compliant ensuring good tissue contact when applied to the treatment site, allowing fluoroscopy to be effectively used and eliminating the need for intra-cardiac echocardiography (ICE) imaging.

The flexible member 38, in one embodiment, is a catheter defining a lumen. The flexible member 38 may be slidably disposed within the lumen of the catheter 28, for example. The catheter 38 may be made from a conductive polymer. Alternatively, the walls of the lumen of the catheter 38 may be coated with a conductive substance. Alternatively, it may be embedded with a metallic conductor. In each case, the conducting portion of the flexible member 38 makes contact with the conformable surface 46. In another embodiment, the flexible member 38 is a solid flexible conductor.

In one embodiment the RF pod 42 is sufficiently rigid to remain expanded when a partial vacuum is drawn on the flexible member 38. In this embodiment the conformable surface 46 of the RF pod 42 includes openings (not shown) that permit fluids adjacent the pod openings to be drawn into the RF pod 42 and up the lumen of the flexible member 38 under vacuum. In this embodiment, the RF pod 42 is drawn by suction to the surface of the heart, e.g., the right atrial septum surrounding the right atrial opening into the tunnel of the PFO and is firmly attached to the surface by the negative pressure within the pod 42.

In another embodiment, the RF pod 42 includes a temperature sensor such as a thermocouple or a thermostat. In still yet another embodiment, the flexible member 38 in the form of a catheter includes an additional lumen that may be used to house, for example, a balloon (not shown).

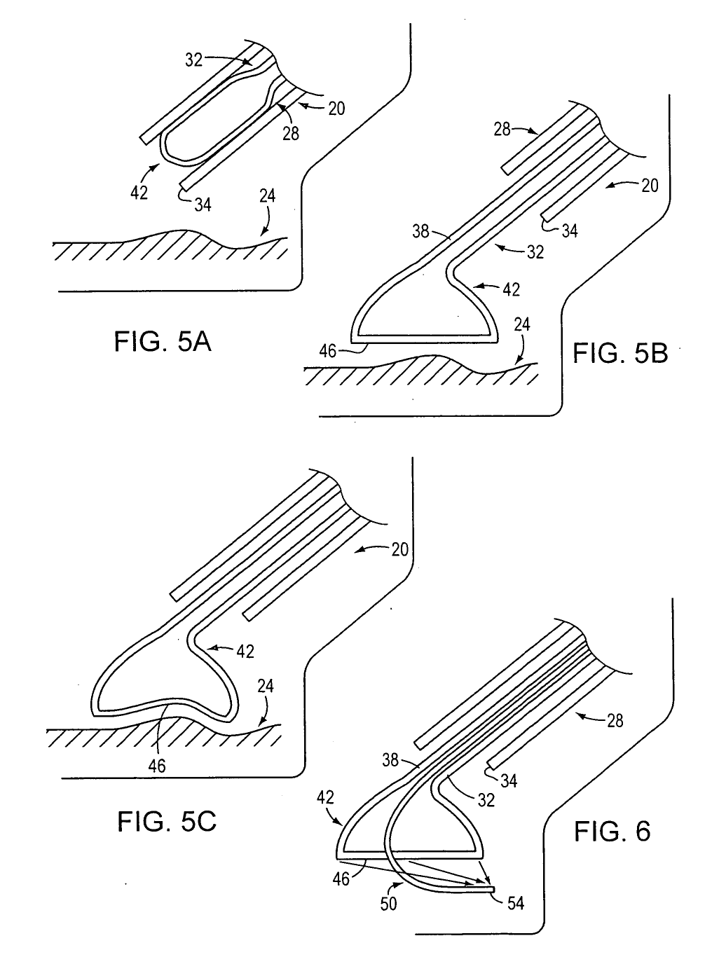

Referring now to FIG. 5a, an embodiment of the apparatus 20 is shown prior to contact with a surface 24 of the heart. The embodiment shown includes a delivery catheter portion 28 and an RF electrode portion 32. In FIG. 5a, the RF pod 42 is positioned within the delivery catheter portion 28 in a collapsed state. The delivery catheter portion 28 is used to bring the RF electrode portion 32 into position within the heart.

When the delivery catheter portion 28 is positioned adjacent the cardiac surface 24, as illustrated in FIG. 5b, the RF electrode portion 32 is pushed out of the distal end 34 of the delivery catheter portion 28, or the delivery catheter 28 is withdrawn proximally from the RF electrode portion 32. The RF pod 42 then expands, orienting the conformable surface 46 to contact the surface of the treatment site 24 in the heart. Referring to FIG. 5c, once in this position, the RF pod 42 is pushed toward the cardiac surface 24 until the conformable surface 46 deforms to interface with the contours of the cardiac surface 24.

Once the conductive conformable surface 46 is positioned against the cardiac surface 24, an RF voltage is applied and current flows through the flexible member 38, the conductive conformable surface 46, through the heart surface 24 to the ground (not shown). Alternatively, as described above with respect to FIG. 3, the RF pod illustrated in FIG. 5a, may include a plurality of pores (not shown) on its conformable surface 46. Saline or other conductive media is used to inflate the RF pod 42. As the RF pod 42 is inflated, the conductive media weeps from through the pores of the conformable surface 46 of the RF pod 42 thereby creating a conductive media interface between the conformable surface 46 and the cardiac tissues 24. In this embodiment, the conductive media serves as the conductor of RF energy to the cardiac tissues.

Referring now to FIG. 6, another embodiment of the invention includes a second electrode 50 in the form of an elongate member, for example, a guidewire, which passes through or adjacent to the flexible member 38 and the conformable surface 46 of the RF pod 42. In one embodiment the elongate member 50 is insulated along its length except for its distal tip 54. The uninsulated tip 54 tends to concentrate the RF energy by having a high density current to flow in the vicinity of the tip 54.

In one embodiment the elongate member 50 is steerable. In another embodiment the region near the tip 54 of the elongate member 50 is a bioabsorbable material and may be left behind in the closed PFO tunnel. In still yet another embodiment the tip 54 region also includes a temperature sensor such as a thermocouple or a thermostat.

In use, the elongate member 50 is advanced distally and positioned in the PFO tunnel. The delivery catheter portion 28 and RF electrode portion 32 are then slid over the elongate member 50 until the RF electrode portion 32 is positioned against the cardiac tissue. Alternatively, the delivery catheter portion 28 and RF electrode portion 32 are positioned first, the elongated member 50 is then advanced to inside of the PFO tunnel. In yet another embodiment, the elongated number 50 is slideably moveable and axially positioned parallel and alongside the RF electrode portion 32. An RF voltage is applied and current flows between the conformable conductive surface 46 and the tip 54 of the elongated member 50. While tissue heating occurs, the elongated member 50 is withdrawn proximally back into the delivery catheter portion 28 causing the PFO tunnel to substantially close from distal to proximal along the withdrawn path of the elongated member 50.

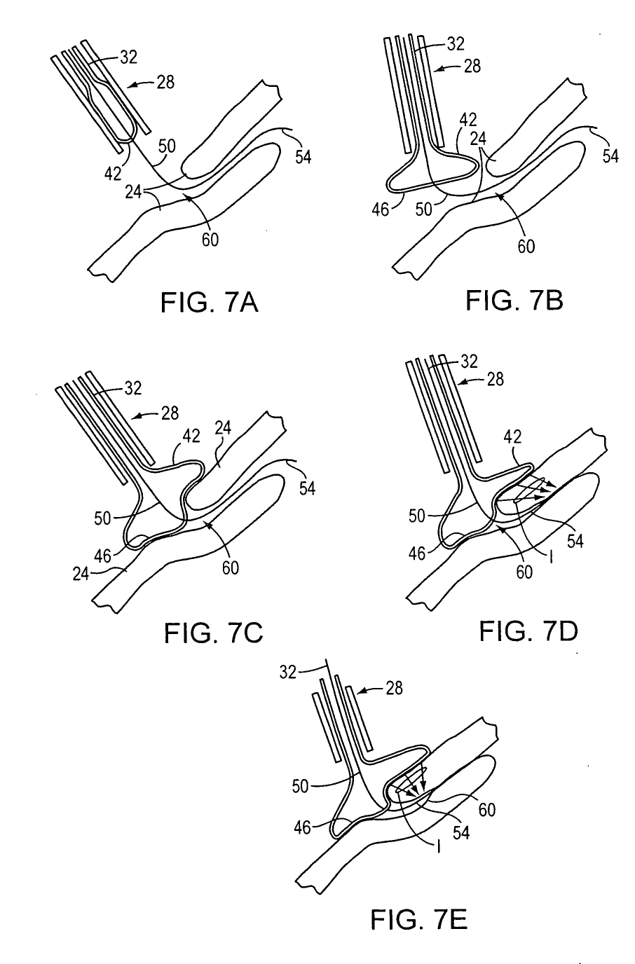

Referring now to FIG. 7a, an embodiment of the invention is shown as the delivery catheter portion 28 with the RF electrode portion 32 prior to contact with a cardiac surface 24, the RF pod 42 is positioned within the delivery catheter portion 28 in a collapsed state. Still referring to FIG. 7a, an elongated member 50 is introduced into the PFO into the heart chamber and has been positioned within the PFO tunnel 60. The elongated member is positioned such that the tip 54 of the elongated member 50 extends through the PFO tunnel 60.

When the delivery catheter portion 28 is positioned adjacent the right cardiac surface of the PFO, as illustrated in FIG. 7b, the RF electrode portion 32 is pushed out of the distal end of the delivery catheter portion 28. The RF pod 42 expands. Alternatively, the delivery catheter 28 is withdrawn proximally from the RF electrode portion 32, and the RF pod 42 expands. The RF electrode portion 32 is advanced further until the conformable conductive surface 46 contacts the right side cardiac surface of the PFO, as illustrated in FIG. 7c.

Still referring to FIG. 7c, once in this position, the RF pod 42 is pushed toward the right side cardiac surface of the PFO until the conformable surface 46 deforms to interface with the contours of the cardiac surface. The elongated member 50 is then slowly withdrawn proximally, such that the tip 54 of the elongated member 50 is positioned within the PFO tunnel. Referring now to FIG. 7d, RF energy is applied to the surface 46 of the RF pod 42, and current (Arrows I) flows from the surface 46 of the RF pod 42 to the tip 54 of the elongated member 50. Because the non-insulated tip 45 of the elongated member 50 is small compared to the surface 46 of the RF pod 42, the current density is increased, and therefore the RF energy is concentrated in the vicinity of the tip 54, causing localized heating of the tissue.

Referring now to FIG. 7e, the elongated member 50 is continuously withdrawn proximally as the RF energy is applied. As the elongated member 50 is withdrawn, the tip 54 moves through the PFO tunnel causing the septum primum and septum secundum to fuse. Therefore, the PFO tunnel is substantially closed by the application of RF power, not just “spot welded”, along the withdrawn path of the tip 54 of the elongated member 50. When the tip 54 exits the right opening of the PFO tunnel, RF power is removed and the elongated member 50 and the RF electrode portion 32 are then further withdrawn proximally back into the lumen of the delivery catheter portion 28. The delivery catheter portion 28 is removed from the heart.

In another embodiment, the apparatus of the invention may further include an implant, for example a septal occluder, that is delivered to a PFO simultaneous with positioning the elongated member to the cardiac tissue. The implant may include one or more materials, for example, bioabsorbable materials such as native animal tissues, for example, devitalized intestinal submucosa.

According to the invention, the RF pod of the apparatus may be a unipolar system where the energy is transferred from the RF pod to a ground. The RF pod and the elongated member of the apparatus of the invention may establish a unipolar system with two electrodes where the energy transferred from both electrodes to a ground, or a bipolar system where the energy is transferred from the pod to the elongated member, or vice versa.

The embodiments of the present invention shown and described herein are exemplary and one skilled in the art will realize that modifications and changes may be made without deviating from the spirit of the invention. The invention is intended to be limited only by the scope of the attached claims.

What is claimed is:

1. An apparatus for closing a tunnel of a patent foramen ovale (PFO), the tunnel having a cardiac tissue surface, comprising:
a catheter comprising a proximal end and a distal end; and
an electrode pod disposed at the distal end of the catheter, the electrode pod comprising a conformable conducting tissue contacting surface and the electrode pod transitionable from a collapsed position to a substantially expanded configuration,
wherein the conformable conducting tissue contacting surface of the electrode pod contacts the surface of the cardiac tissue adjacent to the entrance of the PFO tunnel to deliver energy to substantially close the PFO.

2. The apparatus of claim 1 further comprising a second electrode.

3. The apparatus of claim 2 wherein the second electrode is disposed on the catheter proximal to the electode pod.

4. The apparatus of claim 1 further comprising an elongate member extending between a proximal end and a distal end, the distal end of the elongate member being extendable from the distal end of the catheter.

5. The apparatus of claim 4 wherein the elongate member further comprises a second electrode disposed at the distal end of the elongate member.

6. The apparatus of claim 1 wherein the catheter further comprises a lumen for applying a negative pressure to the tissue surface in contact with the electrode pod.

7. The apparatus of claim 1 further comprising a steerable guidewire.

8. The apparatus of claim 1 further comprising a temperature sensor selected from a thermocouple or a thermistor.

9. The apparatus of claim 4 wherein the distal end of the catheter further comprises a balloon.

10. The apparatus of claim 1 further comprising a bioabsorbable implant.

11. The apparatus of claim 1 wherein the electrode pod is inflatable.

12. The apparatus of claim 1 wherein the electrode pod comprises a conductive sponge.

13. The apparatus of claim 1 wherein the electrode pod comprises a hydrogel blended with conductive materials.

14. The apparatus of claim 1 wherein the electrode pod comprises an expansible polymeric material on the outside surface enclosing a conductive medium.

15. The apparatus of claim 1 wherein the conformable tissue conducting surface of the electrode pod comprises a plurality of pores.

16. A method for closing a tunnel of a patent foramen ovale (PFO), comprising:
advancing a device comprising an energy delivery element in the lumen of the tunnel of the PFO from a first end of the lumen of the tunnel toward a second end of the lumen of the tunnel;
energizing the energy delivery element;
withdrawing the energized energy delivery element from the second end of the lumen of the tunnel toward the first end of the lumen of the PFO tunnel; and,
substantially sealing the tissues in the tunnel of the PFO from the second end of the tunnel to the first end of the tunnel.

17. A method for closing a tunnel of a PFO, the tunnel having a cardiac tissue surface, comprising:
providing an apparatus comprising a catheter having a proximal end and a distal end, an electrode pod disposed at the distal end of the catheter, the electrode pod comprising a conformable conducting tissue contacting surface wherein the conformable conducting tissue contacting surface of the electrode pod uniformly contacts the surface of the cardiac tissue adjacent to the entrance of the PFO tunnel; and,
delivering energy to the cardiac tissue adjacent to the entrance of the PFO tunnel to substantially close the PFO.
